(19) United States
(12) Patent Application Publication
(10) Pub. No.: US 2019/0276495 A1
(43) Pub. Date: Sep. 12, 2019

(54) EBOLA VIRUS VACCINE

(71) Applicants: RIKEN, Saitama (JP); ANIMAL ALLERGY CLINICAL LABORATORIES INC., Sagamihara-shi, Kanagawa (JP)

(72) Inventors: Kenichi MASUDA, Saitama (JP); Takashi SAITO, Saitama (JP); Yasuyuki ISHII, Saitama (JP); Ayato TAKADA, Sapporo-shi (JP); Manabu IGARASHI, Sapporo-shi (JP); Junki MARUYAMA, Sapporo-shi (JP); Yusuke SAITO, Sagamihara-shi (JP); Takuya NARA, Sagamihara-shi (JP)

(73) Assignees: RIKEN, Saitama (JP); ANIMAL ALLERGY CLINICAL LABORATORIES INC., Sagamihara-shi, Kanagawa (JP)

(21) Appl. No.: 16/336,894

(22) PCT Filed: Sep. 27, 2017
(86) PCT No.: PCT/JP2017/034831

§ 371 Date: Mar. 26, 2019

(30) Foreign Application Priority Data
Sep. 27, 2016 (JP) 2016-188897

(51) Int. Cl. C07K 7/08; C07K 7/06; A61K 39/39; A61P 31/14; A61K 39/12

(52) U.S. Cl. CPC C07K 7/08; C07K 7/06; A61K 2039/55572; A61P 31/14; A61K 39/12; A61K 39/39

(57) ABSTRACT

This application provides: a multiple antigen peptide comprising a dendritic core and 4-8 antigen peptides, wherein each of the antigen peptides is bound to a terminus of the dendritic core directly or through a spacer and is a peptide consisting of 7-15 consecutive amino acids in the amino acid sequence of SEQ ID NO: 1, or a peptide which is the same as the peptide except that 1-3 amino acids are substituted; and an immune inducer comprising the multiple antigen peptide(s), wherein the multiple antigen peptide and the immune inducer are both useful for prevention or treatment of Ebola virus infection.

### CROSS-REFERENCE TO RELATED APPLICATIONS

This application is the U.S. National Stage of PCT/JP2017/034831, filed Sep. 27, 2017, which claims priority to JP 2016-188897, filed Sep. 27, 2016.

The instant application contains a Sequence Listing which has been submitted in ASCII format via EFS-WEB and is hereby incorporated by reference in its entirety. Said ASCII copy, created on May 9, 2019, is named sequence.txt and is 16,632 bytes.

### TECHNICAL FIELD

The present invention relates to a multiple antigen peptide (MAP) and an immune inducer comprising the MAP, for use in prevention or treatment of Ebola virus infection.

### BACKGROUND ART

Ebola virus is a minus single-stranded RNA virus and is classified into the genus Ebolavirus of the family Filoviridae. This virus causes serious Ebola hemorrhagic fever in primates such as human, and its lethality is extremely high.

According to information reported by the National Institute of Infectious Diseases (Tokyo, Japan), this virus invades into the body from mucosa or wounds through a body fluid such as blood of a subject and first grows in monocytes/macrophages, dendritic cells, or the like, and then the infection expands to vascular endothelial cells and parenchymal cells of organs in the whole body, and the virus also grows therein, thereby leading to functional disorders of the cells and finally to functional disorders of each organ in the body. It has also been pointed out that, from macrophages infected with Ebola virus, large amounts of various kinds of cytokines may be released, causing failure of the blood coagulation system, plasma leakage, multiple organ failure, and the like. Five phylogenetically different species are known for the genus Ebolavirus: Zaire ebolavirus, Sudan ebolavirus, Reston ebolavirus, Tai forest ebolavirus, and Bundibugyo virus.

According to several studies, administering specific antibodies against surface glycoprotein (GP) of Ebola virus to non-human primates has succeeded in protection against Ebola virus infection. In practical applications to human, all antibodies for treatment whose characteristics have been clarified are GP-specific antibodies.

There are only a small number of peptide vaccines against Ebola virus. For example, an artificial polypeptide having substantially the same antigenicity as GP of Ebola virus (Patent Literature 1), and a liposome to which a peptide having a length of 9-11 amino acids and useful as an Ebola virus vaccine is bound, which peptide has a particular amino acid sequence from Ebola virus and is restricted to HLA-A*0201 (Patent Literature 2), have been reported. For Zaire ebolavirus, it has been reported that Lys114, Lys115, Lys140, Gly143, Pro146, and Cys147 of the Ebola virus GP protein are residues important for invasion of the virus, and that Phe88, Ile113, Pro116, Asp117, Gly118, Ser119, Glu120, Arg136, Tyr137, Val138, His139, Val141, Ser142, Thr144, Gly145, Arg172, and Gly173 are residues involved in the binding to receptors (for example, Non-patent Literature 1). However, no peptide vaccines effective for Ebola virus have been developed.

Furthermore, since antigenicity of GP is different among Ebola virus species, developing a general type of therapeutic antibody (in other words, a therapeutic antibody having wide range of cross-reactivity) has been extremely difficult, and the same applies to development of the vaccines. In Non-patent Literature 2, equal amounts of plasmids for expression of each of GP, matrix protein VP40, and nuclear protein NP were introduced into HEK293T cells, and BALB/c mice of 15 weeks old were immunized with virus-like particles purified from the resulting culture supernatant, to succeed in obtaining a general type of therapeutic antibody for Ebola virus. However, since the antibody is not effective for virus strains that acquire mutations to escape from antibody recognition, it is difficult to obtain similar antibodies effective for the mutant strains again.

In recent years, development of synthetic peptide vaccines has been intensively carried out. In particular, multiple antigen peptides (MAPs) are attracting attention. A MAP peptide can be obtained by using, as a core, a binding substance comprising a plurality of residues of lysine (Lys), which is one of amino acids, and, where needed, a cysteine residue (Cys), and peptides, which are corresponding to a part(s) of an antigen recognized by cells, bind to the α-amino and ε-amino groups of Lys or to the sulfhydryl group of Cys, of the core.

For example, Patent Literature 3 uses a MAP for pneumococcus. Specifically, this document describes that two portions were selected from an antigen peptide of pneumococcus, and that these two kinds of peptides were alternately arranged to prepare a MAP-4 structure, which has a total of four peptides. Preparation of MAPs is also described in Patent Literature 4, Patent Literature 5, and Non-patent Literature 3 to 5.

### CITATION LIST

### Patent Literature

Patent Literature 1: JP 2006-265197 A
Patent Literature 2: JP 2014-005205 A
Patent Literature 3: JP 2011-57691 A
Patent Literature 4: WO 1993/022343 A1
Patent Literature 5: WO 2015/190555 A1

### Non-Patent Literature

Non-patent Literature 1: J. E. Lee and E. O. Saphire, Future Virol. 2009, 4(6): 621-635
Non-patent Literature 2: Wakako Furuyama et al., Scientific Reports 2016, 6: 20514-20523
Non-patent Literature 3: Myron Christodoulides and John E. Heckels, Microbiology 1994, 140: 2951-2960
Non-patent Literature 4: Manju B. Joshi et al., Infection and Immunity 2001, 69: 4884-4890
Non-patent Literature 5: Jon Oscherwitz et al., Infection and Immunigy 2009, 77: 3380-3388

### SUMMARY OF INVENTION

### Problem to be Solved by the Invention

An object of the present invention is to provide a general type of immune inducer (for example, a vaccine) for prevention or treatment of Ebola virus infection using a peptide from Ebola virus.

As described in the BACKGROUND ART section, development of general types of vaccines against Ebola virus that are effective for a plurality of species has been demanded, and, in particular, immune inducers practically applicable to mutant strains have been required. Means for Solution of Problem

The present inventors intensively studied in order to solve the above problem. The present inventors have now found that a particular portion in the amino acid sequences of GPs of various species of Ebola virus is optimal as a general type of antigen peptide, and have developed a multiple antigen peptide having a plurality of molecules of this antigen peptide. As a result, the present inventors have now confirmed production of IgG antibodies against the multiple antigen peptide and have found that an immune inducer applicable as vaccine can be provided using the multiple antigen peptide, thereby completing the present invention.

Specifically, the present invention has the following characteristics.

(1) A multiple antigen peptide comprising a dendritic core and 4-8 antigen peptides, wherein each of the antigen peptides is bound to a terminus of the dendritic core directly or through a spacer, and is a peptide consisting of 7-15 consecutive amino acids in the amino acid sequence of SEQ ID NO: 1, or a peptide which is the same as the peptide except that 1-3 amino acids are substituted.

(2) The multiple antigen peptide according to (1), wherein the peptide is a peptide consisting of 7-15 consecutive amino acids in the amino acid sequences of SEQ ID NOs: 8 to 12, or a peptide which is the same as the peptide except that 1-3 amino acids are substituted.

(3) The multiple antigen peptide according to (1), wherein the peptide is a peptide consisting of 7-15 consecutive amino acids in the amino acid sequence of SEQ ID NO: 2, SEQ ID NO: 3, or SEQ ID NO: 4, or a peptide which is the same as the peptide except that 1-3 amino acids are substituted.

(4) The multiple antigen peptide according to (1) or (3), wherein the peptide is a peptide consisting of 7-11 consecutive amino acids in the amino acid sequence of SEQ ID NO: 5, a peptide consisting of 7 or 8 consecutive amino acids in the amino acid sequence of SEQ ID NO: 32, or a peptide consisting of 7-9 consecutive amino acids in the amino acid sequence of SEQ ID NO: 6, or a peptide which is the same as one of the peptides except that 1-3 amino acids are substituted.

(5) The multiple antigen peptide according to any one of (1) to (4), wherein all of the antigen peptides are peptides consisting of an identical amino acid sequence.

(6) The multiple antigen peptide according to any one of (1) to (5), wherein the dendritic core comprises a plurality of lysine residues.

(7) The multiple antigen peptide according to (6), wherein the dendritic core further comprises a cysteine residue.

(8) The multiple antigen peptide according to any one of (1) to (7), wherein the spacer comprises a polyoxyalkylene chain.

(9) The multiple antigen peptide according to any one of (1) to (8), wherein the multiple antigen peptide is characterized by being represented by the following Formula (I):

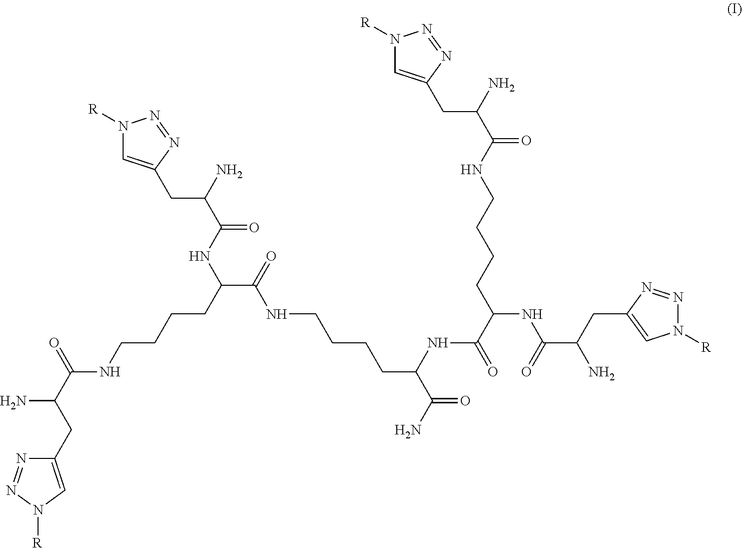

where R is:

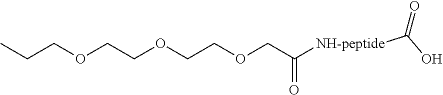

where the peptide represents an antigen peptide.

(10) An immune inducer comprising one or at least two multiple antigen peptides according to any of (1) to (9) as the active ingredient.

(11) The immune inducer according to (10), further comprising an adjuvant having an ability to produce interferon γ.

(12) The immune inducer according to (11), wherein the adjuvant is α-galactosylceramide or an analog thereof.

(13) The immune inducer according to any one of (10) to (12), which is for use in treatment or prevention of Ebola virus infection in a mammal.

(14) The immune inducer according to any one of (10) to (13), comprising a pharmaceutically acceptable carrier.

(15) A method for treatment or prevention of Ebola virus infection in a mammal, comprising administering the immune inducer according to any one of (10) to (14) to the mammal.

According to the present invention, the advantageous effect is achieved that a multiple antigen peptide (MAP) produced by binding peptides of a particular region(s) from surface glycoprotein (GP) of Ebola virus to a dendritic core can increase an IgG antibody titer so as to function as a vaccine in a mammal. Taking it into account that there was no effective method for prevention or treatment of Ebola virus infection, the present invention could be said to have a better effect.

The present description includes the disclosure of Japanese Patent Application No. 2016-188897 from which the present application claims priority.

### BRIEF DESCRIPTION OF DRAWINGS

FIG. 1 shows the MAP structures of MAP-2, MAP-4, MAP-8, and MAP-16.

FIG. 2 shows the results of IgG antibody titers against Ebola 1 when Ebola 1 MAP4 was intravenously administered to mice. In the figure, Group 1 is the results of when administered with physiological saline containing 100 μg of Ebola 1 MAP4 and 10% serum; Group 2 is the results of when administered with physiological saline containing 10 μg of Ebola 1 MAP4 and 10% serum; Group 3 is the results of when administered with physiological saline containing 1 μg of Ebola 1 MAP4 and 10% serum; and Group 4 is the results of when administered with physiological saline containing 100 μg of Ebola 1 MAP4.

FIG. 3 shows the results of IgM antibody titers against Ebola 1 determined when Ebola 1 MAP4 was intraperitoneally administered to mice.

FIG. 4 shows the results of IgG antibody titers against Ebola 1 (left panel) and against Ebola 2 (right panel) determined when Ebola 1 MAP4 and Ebola 2 MAP4 (as a mixture) were simultaneously intraperitoneally administered to mice.

FIG. 5 shows the results of IgM antibody titers against Ebola 1 (left panel) and against Ebola 2 (right panel) determined when Ebola 1 MAP4 and Ebola 2 MAP4 (as a mixture) were simultaneously intraperitoneally administered to mice.

### MODES FOR CARRYING OUT THE INVENTION

The present invention will be described in more detail.

### 1. Multiple Antigen Peptide

According to the first aspect, the present invention provides a multiple antigen peptide comprising a dendritic core and 4-8 antigen peptides, wherein each of the antigen peptides is bound to a terminus of the dendritic core directly or through a spacer and is a peptide consisting of 7-15 consecutive amino acids in the amino acid sequence of SEQ ID NO: 1, or a peptide which is the same as the peptide except that 1-3 amino acids are substituted.

The term “multiple antigen peptide” (MAP) as used herein is a macromolecular substance comprising: a dendritic core having a dendritic polymer (i.e., dendrimer) structure; and a plurality of a same kind or different kinds of peptides from Ebola virus surface glycoprotein, each peptide being bound to a dendritic terminus of the core directly or through a spacer.

The term “a peptide which is the same as the peptide except that 1-3 amino acids are substituted” as used herein means a peptide in which the amino acid that substitutes for each of the 1-3 amino acids in the antigen peptide is any amino acid other than cysteine (Cys), preferably an amino acid having a chemical property (e.g., hydrophobicity, polarity, cationicity, anionicity, electric neutrality, or the like) or structural property (e.g., branch structure, aromaticity, or the like) similar to the substituted amino acid (i.e., the amino acid to be substituted).

The dendritic core is a dendritic supporting core for binding a plurality of, preferably 4-8, peptides (hereinafter, referred to as “antigen peptides” for convenience) from the Ebola virus surface glycoprotein. The dendritic core may have a commonly known structure, and a dendritic polymer basically having two or more identical branches that extend from a core molecule having at least two functional groups may be preferably selected. The dendritic core is also called dendritic polymer. Examples of the dendritic core include, but are not limited to, the structures described in U.S. Pat. Nos. 4,289,872 and 4,515,920. From the viewpoint of its simple production or the like, the dendritic core is preferably a peptide containing a plurality of lysine residues (K). The peptide containing lysine residues may also contain a cysteine residue (C). For example, in case of a K-K-K structure, which comprises three lysine residues (K), one molecule of the antigen peptide may be bound to each of the α-amino group side and the ε-amino group side of the lysine residue (K) at each terminus. In this case, at most 4 antigen peptides can be bound to the K-K-K structure. To the lysine residue (K), a spacer peptide may be bound through α-carboxyl group of the lysine residue. The spacer peptide is preferably a peptide consisting of 2-10 amino acid residues, such as K-K-C or K-βA-C, where βA represents a β-alanine residue and C represents a cysteine residue. When the amino acid residue at the N-terminus of the spacer peptide is, for example, a lysine residue (K), a K-K-K structure with at most 4 antigen peptides bound thereto as described above may be linked to the amino acid residue through α-amino group of the lysine residue. In such case, the prepared MAP has at most 8 antigen peptides.

According to the present invention, the antigen peptides are from glycoproteins of an Ebola virus.

Depending on the areas in which Ebola virus infection occurred, the virus has been reported as Zaire ebolavirus, Sudan ebolavirus, Reston ebolavirus, Tai forest ebolavirus, and Bundibugyo virus. The examples of the amino acid sequences of glycoproteins from the above-exemplified Ebola viruses are described in, for example, KR534526 (Zaire ebolavirus), FJ968794 (Sudan ebolavirus), NC_004161 (Reston ebolavirus), FJ217162 (Tai forest ebolavirus), and KR063673 (Bundibugyo ebolavirus), as GenBank accession numbers of NCBI, USA.

For example, the nucleotide numbers 5900-8305 of the genomic glycoprotein gene (KR534526) of Zaire ebolavirus encodes a spike glycoprotein precursor (SEQ ID NO: 7). Amino acid sequences of the glycoproteins from other species of Ebola virus corresponding to the sequence of the spike glycoprotein precursor and nucleotide sequences encoding them, are described as, for example, GenBank accession numbers FJ968794, NC_004161, FJ217162, and KR063673.

SEQ ID NO: 7:
MGVTGILQLPRDRFKRTSFFLWVIILFQRTFSIPLGVIHNSTLQVSDVD
KLVCRDKLSSTNQLRSVGLNLEGNGVATDVPSVTKRWGFRSGVPPKVVN
YEAGEWAENCYNLEIKKPDGSECLPAAPDGIRGFPRCRYVHKVSGTGPC
AGDFAFHKEGAFFLYDRLASTVIYRGTTFAEGVVAFLILPQAKKDFFSS
HPLREPVNATEDPSSGYYSTTIRYQATGFGTNEAEYLFEVDNLTYVQLE
SRFTPQFLLQLNETIYASGKRSNTTGKLIWKVNPEIDTTIGEWAFWETK
KNLTRKIRSEELSFTAVSNGPKNISGQSPARTSSDPETNTTNEDHKIMA
SENSSAMVQVHSQGRKAAVSHLTTLATISTSPQPPTTKTGPDNSTHNTP
VYKLDISEATQVGQHHRRADNDSTASDTPPATTAAGPLKAENTNTSKSA
DSLDLATTTSPQNYSETAGNNNTHHQDTGEESASSGKLGLITNTIAGVA
GLITGGRRTRREVIVNAQPKCNPNLHYWTTQDEGAAIGLAWIPYFGPAA
EGIYTEGLMHNQDGLICGLRQLANETTQALQLFLRATTELRTFSILNRK
AIDFLLQRWGGTCHILGPDCCIEPHDWTKNITDKIDQIIHDFVDKTLPD
QGDNDNWWTGWRQWIPAGIGVTGVIIAVIALFCICKFVF

The sequence of amino acid numbers 110-147 in the amino acid sequence of SEQ ID NO: 7, that is, NLEIKKPDGSECLPAAPDGIRGFPRCRYVHKVSGTGPC (SEQ ID NO: 8), is an example of the amino acid sequence of SEQ ID NO: 1:

NL(Xaa=E, A, or D)IKK(Xaa=P, S, V, or A)DGSECLP(Xaa=A, P, L, or EXXaa=A or P)P(Xaa=D or E)G(Xaa=I or V)R(Xaa=G or D)FPRCRYVHK(Xaa=V or AXXaa=S or Q)GTGPC. The following amino acid sequences of SEQ ID NOs: 9 to 12 are other examples of the amino acid sequence of SEQ ID NO: 1.

SEQ ID NO: 9:
NLEIKKPDGSECLPPPPDGVRGFPRCRYVHKAQGTGPC
SEQ ID NO: 10:
NLEIKKSDGSECLPLPPDGVRGFPRCRYVHKVQGTGPC
SEQ ID NO: 11:
NLAIKKVDGSECLPEAPEGVRDFPRCRYVHKVSGTGPC
SEQ ID NO: 12:
NLDIKKADGSECLPEAPEGVRGFPRCRYVHKVSGTGPC

By RNA editing of the Ebola virus GP gene, Ebola virus produces three kinds of glycoproteins, i.e., non-structural soluble glycoprotein (sGP), small non-structural soluble glycoprotein (ssGP), and surface glycoprotein GP. The surface glycoprotein GP forms homotrimer spikes, and is responsible for membrane fusion between the cell membrane and the viral envelope in binding to a receptor of a target cell (i.e., viral entry). The GP is therefore important for the life cycle and the induction of pathogenicity of the virus. Among the three kinds of glycoproteins of Ebola virus, whether or not sGP and ssGP, which are non-structural and soluble (or secretory) glycoproteins, play important roles in the pathogenicity of the virus has not been fully clarified, although the surface glycoprotein GP has the same sequence as the amino acid sequence on the N-terminal sides of sGP and ssGP, which sequence comprises the amino acid sequence of SEQ ID NO: 1. The trimer GP, which is an Ebola virus particle surface glycoprotein, is important for the life cycle of the virus and is involved in differences in pathogenicity among viral strains.

Focusing on the amino acid sequence of SEQ ID NO: 1 in the Ebola virus GP proteins, the multiple antigen peptide of the present invention can provide an immune inducer comprising, as an antigen peptide, a peptide consisting of 7-15 consecutive amino acids in the amino acid sequence of SEQ ID NO: 1 (for example, the amino acid sequences of SEQ ID NOs: 8 to 12) shared by all of the three kinds of glycoproteins, or a peptide having the same amino acid as this peptide except that 1-3 amino acids are substituted, which immune inducer can be also used as a vaccine against Ebola virus infection when a same kind or different kinds, preferably a same kind, of a plurality of antigen peptides are bound to the dendritic core as described above.

The following are further examples of the antigen peptides of the present invention. The antigen peptides are, however, not limited to these peptides.

The first examples are peptides consisting of 7-15 consecutive amino acids, preferably 9-12 consecutive amino acids, in the amino acid sequences of SEQ ID NOs: 2 and 13 to 17, which sequences correspond to amino acid numbers 110-126 of the amino acid sequence of SEQ ID NO: 7).

SEQ ID NO: 2:
NL(Xaa = E, A, or D)IKK(Xaa = P, S, V, or A)
DGSECLP(Xaa = A, P, L, or E)(Xaa = A or P)P
SEQ ID NO: 13:
NLEIKKPDGSECLPAAP
SEQ ID NO: 14:
NLEIKKPDGSECLPPPP
SEQ ID NO: 15:
NLEIKKSDGSECLPLPP
SEQ ID NO: 16:
NLAIKKVDGSECLPEAP
SEQ ID NO: 17:
NLDIKKADGSECLPEAP

The second examples are peptides consisting of 7-15 consecutive amino acids, preferably 9-12 consecutive amino acids, in the amino acid sequences of SEQ ID NOs: 3 and 18 to 22, which sequences correspond to amino acid numbers 126-143 of the amino acid sequence of SEQ ID NO: 7.

SEQ ID NO: 3:
P(Xaa = D or E)G(Xaa = I or V)R(Xaa = G or D)
FPRCRYVHK(Xaa = V or A)(Xaa = S or Q)G
SEQ ID NO: 18:
PDGIRGFPRCRYVHKVSG
SEQ ID NO: 19:
PDGVRGFPRCRYVHKAQG
SEQ ID NO: 20:
PDGVRGFPRCRYVHKVQG
SEQ ID NO: 21:
PEGVRDFPRCRYVHKVSG
SEQ ID NO: 22:
PEGVRGFPRCRYVHKVSG

The third examples are peptides consisting of 7-15 consecutive amino acids, preferably 9-12 consecutive amino acids, in the amino acid sequences of SEQ ID NOs: 4 and 23 to 27, which sequences correspond to amino acid numbers 130-147 of the amino acid sequence of SEQ ID NO: 7.

SEQ ID NO: 4:
R(Xaa = G or D)FPRCRYVHK(Xaa = V or A)
(Xaa = S or Q)GTGPC
SEQ ID NO: 23:
RGFPRCRYVHKVSGTGPC
SEQ ID NO: 24:
RGFPRCRYVHKAQGTGPC
SEQ ID NO: 25:
RGFPRCRYVHKVQGTGPC
SEQ ID NO: 26:
RDFPRCRYVHKVSGTGPC
SEQ ID NO: 27:
RGFPRCRYVHKVSGTGPC

The fourth examples are peptides consisting of 7-11 consecutive amino acids in the amino acid sequences of SEQ ID NOs: 5 and 28 to 31, which sequences correspond to amino acid numbers 113-123 of the amino acid sequence of SEQ ID NO: 7.

SEQ ID NO: 5:
IKK(Xaa = P, S, V, or A)DGSECLP
SEQ ID NO: 28:
IKKPDGSECLP
SEQ ID NO: 29:
IKKSDGSECLP
SEQ ID NO: 30:
IKKVDGSECLP
SEQ ID NO: 31:
IKKADGSECLP

The fifth examples are peptides consisting of 7-9 consecutive amino acids in the amino acid sequence of SEQ ID NO: 6, which sequence corresponds to amino acid numbers 132-140 of the amino acid sequence of SEQ ID NO: 7.

SEQ ID NO: 6:
FPRCRYVHK

The sixth examples are peptides consisting of 7-8 consecutive amino acids in the amino acid sequences of SEQ ID NOs: 32-36, which sequences correspond to amino acid numbers 126-133 of the amino acid sequence of SEQ ID NO: 7.

SEQ ID NO: 32:
P(Xaa = D or E)G(Xaa = I or V)R(Xaa = G or D)FP
SEQ ID NO: 33:
PDGIRGFP
SEQ ID NO: 34:
PDGVRGFP
SEQ ID NO: 35:
PEGVRDFP
SEQ ID NO: 36:
PEGVRGFP

The present invention also provides an antigen peptide selected from the group consisting of peptides of the amino acid sequences of SEQ ID NOs: 1, 2, 3, and 4, and an antigen peptide of an amino acid sequence selected from the group consisting of the amino acid sequences of SEQ ID NOs: 5, 6, and 8 to 36.

The antigen peptides constituting the multiple antigen peptide (MAP) of the present invention are bound to the termini of the dendritic core directly or through a spacer, wherein preferably the antigen peptides are covalently bound to each terminus of the dendritic core one by one. For example, a functionalized dendritic core may be bound to a functionalized solid-phase resin, and a reactive functional group of each antigen peptide may be reacted with and bound to the reactive functional group at the dendritic terminus (W. Kowalczyk et al., J. Pep. Sci. 2011, 17: 247-251). In this case, the antigen peptides may be synthesized by known techniques including synthesizing by use of an automated peptide synthesizer based on predetermined amino acid sequences (for example, J. M. Stewart and J. D. Young, Solid Phase Peptide Synthesis, 2d ed., Pierce Chemical Company, 1984; and G. B. Fields et al., Principles and Practice of Peptide Synthesis, in G. A. Grant (ed.): Synthetic Peptides: A User's Guide, W.H. Freeman, 1992). Alternatively, the antigen peptides may be prepared by using known DNA recombination techniques (for example, M. R. Green and J. Sambrook, Molecular Cloning A Laboratory Manual, Vol. 1 and Vol. 2, Cold Spring Horbor Laboratory Press, fourth edition, 2012).

The MAP of the present invention comprises a plurality of, preferably 2-16 and more preferably 4-8, antigen peptides, and the antigen peptides may be of a same kind or different kinds, preferably of a same kind. As used herein, the term “same kind of antigen peptide” means a peptide having the same epitope properties, which peptide includes a peptide having a high identity. The “peptide having a high identity” is a peptide having substitution of 1-3 amino acids, preferably 1 or 2 amino acids, more preferably a single (1) amino acid, relative to any one of a plurality of antigen peptides. The amino acid(s) that substitutes an amino acid(s) in the antigen peptide is any amino acid except for cysteine (Cys), preferably an amino acid that has a similar chemical property (e.g., hydrophobicity, polarity, cationicity, anionicity, electric neutrality, or the like) or a structural property (e.g., branch structure, aromaticity, or the like) to the substituted amino acid. As used herein, the term “same epitope properties” means properties that can induce in vivo production of an IgG antibody capable of binding to a target protein or polypeptide of interest and of inducing immunity against the virus. When different kinds (that is, not “a same kind”) of antigen peptides are used, at least one of each different antigen peptide is bound to the dendritic core.

The term “subject to which the multiple antigen peptide is administered” (hereinafter also referred to as “subject”) as used herein includes mammals such as humans, domestic animals (e.g., cows, pigs, camels, and the like), pet animals (e.g., dogs, cats, and the like), racing animals (e.g., horses and the like), and animals kept in zoos. The subject is preferably a human.

The MAP of the present invention induces production of class-switched antibodies in the body of a subject. The antibodies produced in the present invention are IgG, IgA, or IgE, preferably IgG. In general, when a foreign substance invades into the body, IgM antibody is produced from B2 B cells within about one week to allow initial protection to function in the body. Since IgM, however, has a short half-life, its antibody titer in blood decreases within about 1 week to 10 days. Following the production of IgM, activation of T cells reactive to the foreign substance occurs gradually in the body, resulting in producing IgG antibodies so as to enhance protection by humoral immunity. Once IgG antibodies are produced, because their half-lives are long, their antibody titers in blood are maintained over a period of from several weeks to several months or longer.

In another aspect, the MAP of the present invention is capable of stimulating B cells in the innate immune system (B1 B cells) to cause production of IgM for a longer period compared to the production by B2 B cells. IgM increased in blood by administration of the MAP of the present invention is confirmed for, for example, 14 days or longer, preferably 21 days or longer.

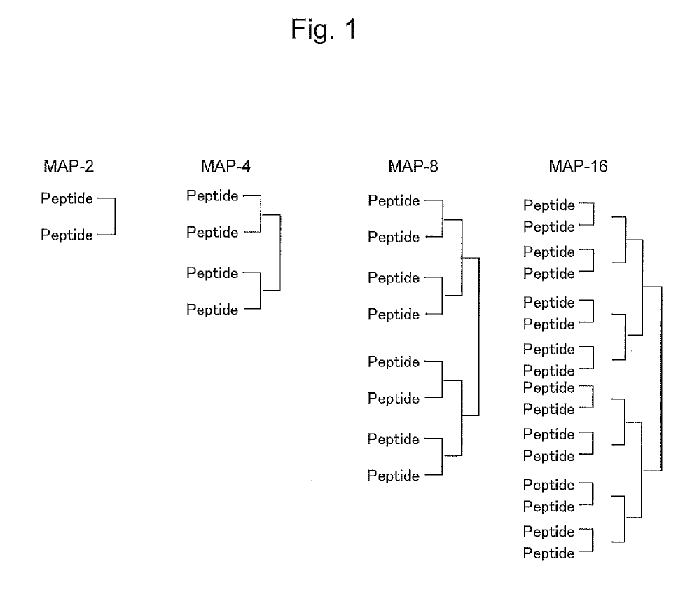

The MAP of the present invention has, for example, the structure shown in FIG. 1. In particular, the MAP has a dendritic structure comprising 4-8 antigen peptides, preferably the same antigen peptides, as shown as MAP-4 or MAP-8. Specifically, the MAP may have the MAP-4 structure of the following Formula (I), but is not limited to thereto.

MAP of Formula I:

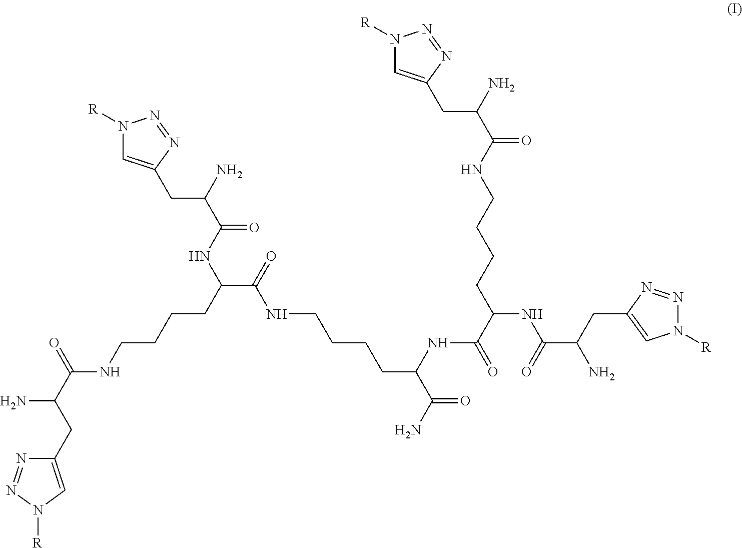

where R is:

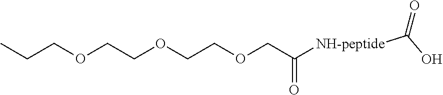

where the peptide represents an antigen peptide.

### 2. Production of Multiple Antigen Peptide (MAP)

The MAP of the present invention can be prepared by a method comprising, for example, the following steps of:

(1) providing a dendritic core having reactive functional groups;

(2) providing a plurality of a same kind or different kinds of antigen peptides each having a reactive functional group;

(3) producing a multiple antigen peptide by reaction of binding the reactive functional group of the dendritic core to the reactive functional group of each antigen peptide; and (4) recovering or collecting the multiple antigen peptide.

As described above, the dendritic core is a dendritic supporting core to bind a plurality of a same kind or different kinds, preferably a same kind, of the antigen peptides, preferably a same kind of (preferably, identical) 4-8 antigen peptides. The dendritic core may have a commonly known structure. The dendritic core preferably comprises a plurality of lysine residues (K), and may also contain a cysteine residue (C). As shown in the exemplified structures of the MAP of the invention in FIG. 1 (preferably, a structure such as MAP-4 or MAP-8), the portions other than the 4-8 antigen peptides are formed by the dendritic core. The dendritic core preferably comprises, for example, a K-K-K sequence for MAP-4, or comprises, for example, a K-K-K-K-K sequence for MAP-8. Usually, a spacer peptide is bound to the K in the center of these sequences. Preferably, the spacer peptide is a peptide consisting of two or more amino acid residues, such as K-K-C or K-βA-C (where βA represents a β-alanine residue), but is not limited to them. The K or K-K in each of the left and right, other than the K in the center, is designed such that two antigen peptides are bound per one K. A spacer may be arranged between the dendritic core and each peptide. The spacer is preferably a highly hydrophilic group containing a polyoxyalkylene chain (e.g., polyoxyethylene chain or polyoxypropylene chain). The number of repeats of oxyalkylene units in the polyoxyalkylene chain is 2 or more, preferably 2-50, more preferably 3-30.

Each terminus of the dendritic core may have an appropriate functional group for binding to the antigen peptide. The functional group is not limited as long as it can be used for modification of a protein, examples of which include amino group, sulfhydryl group, acetylene group, and N-hydroxysuccinimidyl group.

On the other hand, the functional group in the antigen-peptide side is any functional group that is capable of undergoing binding reaction with a terminal functional group of the dendritic core. Examples of this functional group include an N-hydroxysuccinimidyl group for amino group, a sulfhydryl group or carboxyl group for sulfhydryl group, and an azide group for acetylene group. The antigen peptide is as described above.

According to an embodiment of the present invention, a dendritic core having the K-K-K sequence has the following structure, where each terminal functional group has an acetylene group.

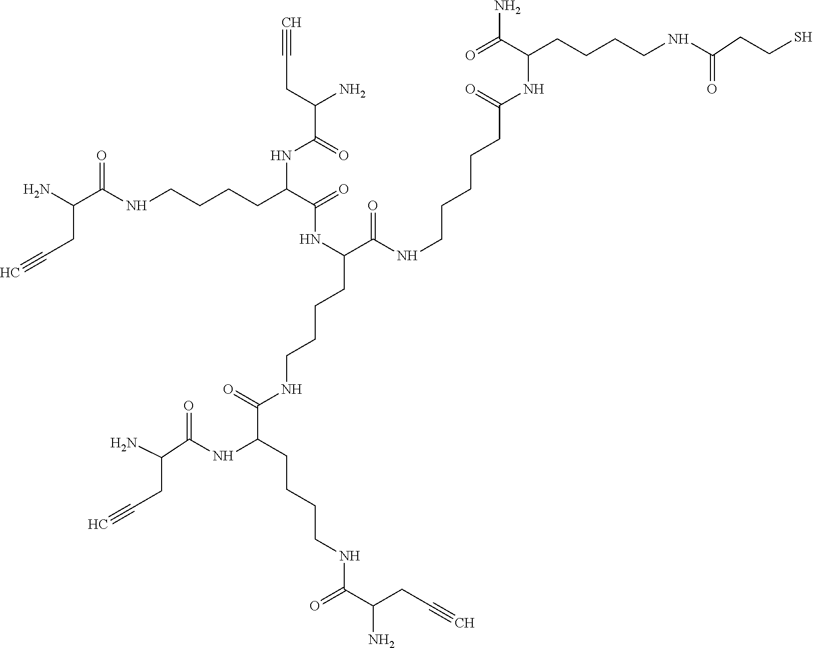 [SMILES: C#CCC(N)C(=O)NCCCCC(NC(=O)C(N)CC#C)C(=O)NCCCCC(NC(=O)C(CCCCNC(=O)C(N)CC#C)NC(=O)C(N)CC#C)C(=O)NCCCCCC(=O)NC(CCCCNC(=O)CCS)C(N)=O]

The terminal functional group of antigen peptide that reacts with the acetylene group in the above structure is an azide group. In this case, the binding reaction i is the Huisgen reaction below. In the following formula, R₁ represents a dendritic core portion, and R₂ represents an antigen peptide.

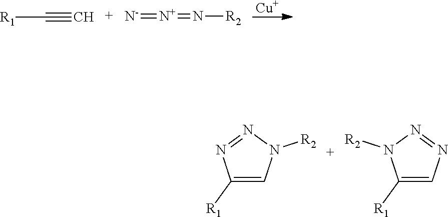

This reaction is a reaction in which an alkyne is bound to an azide by using a monovalent copper ion as a catalyst, and the reaction product is said to be stable and to hardly show side reactions. This reaction is thus attracting attention as the click chemistry. The copper ion catalyst solution can be prepared using an aqueous copper sulfate pentahydrate solution and ascorbic acid.

In the step of recovering the MAP, the peptide is purified. The method for recovering the peptide may be any of common methods to purify proteins or polypeptides, and may be carried out using, individually or in combination, chromatography techniques such as gel filtration chromatography, ion-exchange chromatography, hydrophobic interaction chromatography, reversed-phase chromatography, affinity chromatography, and high-performance liquid chromatography (HPLC). Identification of the peptide of interest can be carried out by nuclear magnetic resonance spectroscopy (NMR), mass spectrum analysis, LC/MS, amino acid analysis, or the like.

### 3. Immune Inducer

The present invention also provides an immune inducer comprising one or at least two multiple antigen peptides (MAPs) described above. The immune inducer of the invention is a preparation that induces production of IgG antibody, or IgG and IgM antibodies.

The immune inducer of the present invention as a pharmaceutical composition can be used for prevention or treatment or amelioration of Ebola virus infection by inducing production of IgG antibodies against the infection, and can also be used as a “vaccine”.

The immune inducer of the present invention, as a pharmaceutical composition, can sustain production of IgM antibody against Ebola virus infection for a long period, and also enables prevention of the infection in uninfected individuals. In addition, by production of IgM antibody for a long period, the immune inducer can be used for prevention of transmission of the infection from infected individuals to uninfected individuals.

The effective dose of the MAP of the present invention per administration in humans is, but is not limited to, for example about 0.05-2.5 μg/kg body weight to 1-10 mg/kg body weight for MAP-4, or, for example 0.5-25.0 μg/kg body weight to 1-10 mg/kg body weight for MAP-8. Herein, the dose may be appropriately changed depending on body weights, ages, sexes, symptoms, severities, administration methods, and the like, of subjects including human.

The immune inducer of the present invention may be in the form of, for example, solutions, suspensions, tablets, injection solutions, granules, emulsions, nebulas, or the like, and may appropriately contain an additive(s) such as vehicle, diluent, binder, disintegrator, lubricant, solubilizer, preservative, flavor, surfactant, and the like. An Adjuvant is basically not needed as long as production of interferon γ can be seen in a subject that the immune inducer is administered to, but it may be added where needed.

The immune inducer of the present invention may comprise an adjuvant. The adjuvant is appropriately selected depending on isotypes of desired antibodies. For example, where IgG is preferentially produced, the adjuvant is a substance that predominantly induces production of interferon 7. The substance that induces production of interferon γ includes, but is not limited to, for example α-galactosylceramide, α-galactosylceramide analogs, and bacterial oligonucleotide CpG. Examples of the α-galactosylceramide analogs include, but are not limited to, compounds described in WO 2007/099999 (U.S. Pat. No. 8,163,705 B), WO 2009/119692 (U.S. Pat. No. 8,551,959 B), WO 2008/102888 (U.S. Pat. No. 8,299,223 B), WO 2010/030012 (U.S. Pat. No. 8,580,751 B), WO 2011/096536 (U.S. Pat. No. 8,853,173 B), and WO 2013/162016 (US 2015-0152128 A). In addition, similarly to the adjuvant, interferon γ may be included for enhancement of the effect of an IgG antibody induction by the immune inducer of the present invention.

The immune inducer of the present invention, as a pharmaceutical composition, can be used for prevention of Ebola virus infection, prevention of spread of the infection, or treatment of the infection.

Thus, the present invention also provides a method for prevention or treatment of the above-described disease, comprising administering the above MAP or the above immune inducer to a subject. In this method, the production of antibodies includes production of IgG antibody, and may also include production of IgM antibody. The production of the antibody in the method of the invention may be carried out for the purpose of treatment or prevention of Ebola virus infection, or prevention of spread of the infection.

Examples of administration routes include, but are not limited to, intravenous administration, intraarterial administration, nasal administration, transmucosal administration, intraperitoneal administration, rectal administration, subcutaneous administration, intramuscular administration, and oral administration.

The immune inducer of the present invention may be prepared by further containing a pharmaceutically acceptable carrier.

The term “pharmaceutically acceptable” has a meaning commonly used in the pharmaceutical industry, and indicates, in some cases, that a substance, composition or the like can be used without causing allergic reactions or similar harmful reactions when it is administered to humans. Preparation of an aqueous composition comprising a protein as the active ingredient is sufficiently understood in the art. Such a composition may be prepared typically as an injection solution, liquid solution, or suspension, and may also be prepared as a solid formulation suitable for dissolution or suspension in the liquid before injection. The prepared product may also be emulsified.

The “carrier” includes any or all solvents, dispersion media, vehicles, coatings, diluents, antibacterial and antifungal agents, isotonic and absorption delaying agents, buffers, carrier solutions, suspensions, and colloids. Examples of the carrier include: buffers of phosphate, citrate, and other organic acid salts; antioxidants containing ascorbic acid; low molecular weight polypeptides (having less than about 10 amino acid residues); proteins (for example, serum albumin, gelatin, or immunoglobulins); hydrophobic polymers (for example, polyvinylpyrrolidone); amino acids (for example, glycine, glutamine, asparagine, arginine, or lysine); monosaccharides, disaccharides, and other carbohydrates such as glucose, mannose, or dextran; chelating agents such as EDTA; sugar alcohols such as mannitol or sorbitol; salt-forming counter ions such as sodium; and/or nonionic surfactants (for example, polyoxyalkylene-based surfactants). Use of the media and substances for pharmaceutically active substances is well known in the art. It is expected that any conventional medium or substance can be used in therapeutic compositions as long as the medium or substance is not incompatible as an active ingredient. An auxiliary active ingredient may also be incorporated in the composition.

For the immune inducer of the present invention, various surfactants used for preparation may be used. The types of the surfactants include, but are not limited to, nonionic surfactants, cationic surfactants, anionic surfactants, and amphoteric surfactants, preferably nonionic surfactants. Examples of the nonionic surfactants include: polyoxyalkylene-based nonionic surfactants such as polyoxyethylene monoalkyl ethers or polyoxyethylene monoaryl ethers; higher fatty acid esters of polyols (for example, sorbitan and sorbitol); and products prepared by addition of ethylene oxide to higher fatty acid esters of polyols by polymerization.

The immune inducer of the present invention may further comprise one or more additional components. Examples of the additional components include, but are not limited to, suspending agents, stabilizers, and dispersants. For stabilization of the immune inducer of the invention, the isoelectric point of the MAP may be lowered to improve its metabolic stability. Specifically, an acidic amino acid(s) (for example, asparagine or glutamic acid) and/or a deoxynucleotide(s) (for example, GpC oligonucleotide or CpG oligonucleotide) may be further contained in the immune inducer. In one embodiment, an acidic amino acid(s) and/or a deoxyoligonucleotide(s) may be directly bound to the MAP of the present invention.

### EXAMPLES

The present invention will be described in more detail by referring to the following Examples. However, the scope of the present invention is not limited by these Examples.

### Example 1

### <Structure of Multiple Antigen Peptide (MAP)>

As the structure of the MAP, the structure of the following Formula (I) was used.

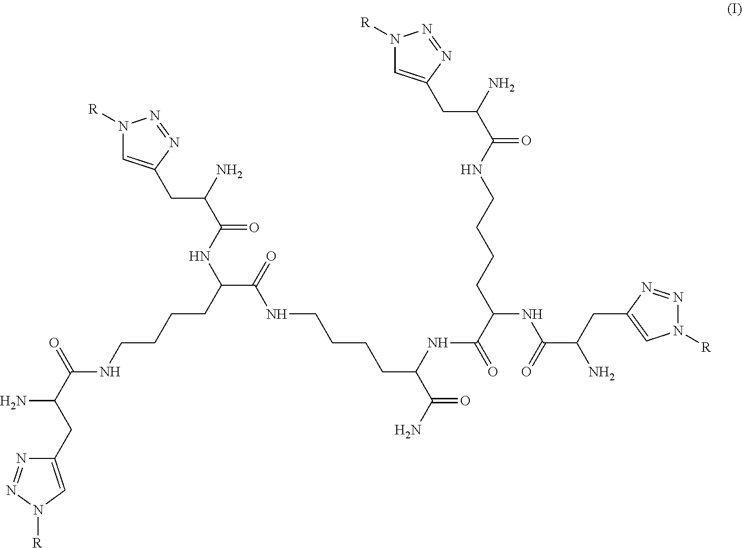

where R is:

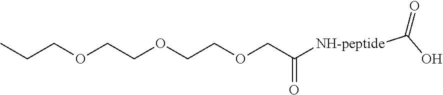

where the peptide represents an antigen peptide.

Four kinds of peptides from glycoproteins of Ebola virus, i.e., IKKADGSECLP (SEQ ID NO: 31), IKKADGSEC(Boc-Cys-OH)LP (SEQ ID NO: 37), FPRCRYVHK (SEQ ID NO: 6), and FPRC(Boc-Cys-OH)RYVHK (SEQ ID NO: 38) were selected and used as antigen peptides for preparation of the MAP.

### <Synthesis of Ebola 1 MAP4>

### 1. Abbreviations

NH2-SAL-Trt(2-Cl)-Resin: Rink-Bernatowitz-amide Barlos Resin (Watanabe Chemical Industries, Ltd., Hiroshima, Japan)

Fmoc-Lys(Fmoc)-OH: N-α,N-ε-Bis(9-fluorenylmethoxycarbonyl)-L-lysine (Watanabe Chemical Industries, Ltd.)

Boc-Pra-OH: N-Boc-L-propargylglycine (Tokyo Chemical Industry Co., Ltd., Tokyo, Japan)

N₃-PEG-COOH: 11-Azido-3,6,9-trioxaundecanoic Acid (Tokyo Chemical Industry Co., Ltd.)

Fmoc-Pro-TrtA-PEG-Resin: N-α-(9-Fluorenylmethoxycarbonyl)-L-proline tritylcarboxamidomethyl polyethyleneglycol resin (Watanabe Chemical Industries, Ltd.)

Fmoc-Ala-OH: N-α-(9-Fluorenylmethoxycarbonyl)-L-alanine (Watanabe Chemical Industries, Ltd.)

Fmoc-Cys(Trt)-OH: N-α-(9-Fluorenylmethoxycarbonyl)-S-trityl-L-cysteine (Watanabe Chemical Industries, Ltd.)

Fmoc-Asp(OtBu)-OH: N-α-(9-Fluorenylmethoxycarbonyl)-L-aspartic acid β-t-butyl ester (Watanabe Chemical Industries, Ltd.)

Fmoc-Glu(OtBu)-OH: N-α-(9-Fluorenylmethoxycarbonyl)-L-glutamic acid γ-t-butyl ester (Watanabe Chemical Industries, Ltd.)

Fmoc-Gly-OH: N-α-(9-Fluorenylmethoxycarbonyl)-glycine (Watanabe Chemical Industries, Ltd.)

Fmoc-Ile-OH: N-α-(9-Fluorenylmethoxycarbonyl)-L-isoleucine (Watanabe Chemical Industries, Ltd.)

Fmoc-Lys(Boc)-OH: N-α-(9-Fluorenylmethoxycarbonyl)-N-ε-(t-butoxycarbonyl)-L-lysine (Watanabe Chemical Industries, Ltd.)

Fmoc-Ile-OH: N-α-(9-Fluorenylmethoxycarbonyl)-L-leucine (Watanabe Chemical Industries, Ltd.)

Fmoc-Pro-OH: N-α-(9-Fluorenylmethoxycarbonyl)-L-proline (Watanabe Chemical Industries, Ltd.)

Fmoc-Ser(tBu)-OH: N-α-(9-Fluorenylmethoxycarbonyl)-O-(t-butyl)-L-serine (Watanabe Chemical Industries, Ltd.)

Boc-Cys(Npys)-OH: N-α-(t-Butoxycarbonyl)-S-(3-nitro-2-pyridinesulfenyl)-L-cysteine (Kokusan Chemical Co., Ltd., Tokyo, Japan)

HATU: O-(7-aza-1H-benzotriazol-1-yl)-N,N,N′,N′-tetramethyluronium hexafluorophosphate (Genscript)

DIEA: N,N-Diisopropylethylamine (Wako Pure Chemical Industries, Ltd. (Osaka, Japan)—for peptide synthesis)

DMF: N,N-Dimethylformamide (Kanto Chemical Co., Inc. (Tokyo, Japan); for peptide synthesis)

TFA: 2,2,2-Trifluoroacetic acid (Wako Pure Chemical Industries, Ltd.)

TIPS: Triisopropylsilane (Watanabe Chemical Industries, Ltd.)

Thioanisole (Watanabe Chemical Industries, Ltd.)

m-Cresol (Tokyo Chemical Industry Co., Ltd.)

DCM: Dichloromethane (Kanto Chemical Co., Inc.)

ACN: Acetonitrile (Kanto Chemical Co., Inc.; for HPLC)

α-CHCA: α-Cyano-4-hydroxycinnamic acid

Preparative column: YMC-Pack Pro C18; 20 mm (I. D.)×250 mm (length); particle size, 5 μm; pore size, 12 μm (YMC)

Analytical column: YMC-Pack Pro C18; 4.6 mm (I. D.)×250 mm (length); particle size, 5 μm; pore size, 12 μm (YMC)

MALDI-TOF MASS: Matrix Assisted Laser Desorption Ionization Time Of Flight Mass Spectrometry

D. W.: Distilled water

Copper sulfate pentahydrate (Kanto Chemical Co., Inc.)

Ascorbic acid (Kanto Chemical Co., Inc.)

### 2. Synthesis of MAP Core

Using MAP4 as an example, a synthesis method for the MAP core is described below.

For the synthesis of the MAP core, conventional Fmoc solid-phase synthesis was employed, and all steps were manually carried out. Specifically, 1 mmol NH2-SAL-Trt(2-Cl)-Resin was used as a solid-phase carrier to perform the synthesis by the following procedures.

TABLE 1

| | | Amino acid | Reaction Time | |
|---|---|---|---|---|
| | Step | (mmol) | (minutes) | Times |
| 1 | Deblock | — | 7 | 1 |
| 2 | Fmoc-Lys(Fmoc)—OH | 3 | 15 | 1 |
| 3 | Deblock | — | 7 | 1 |
| 4 | Fmoc-Lys(Fmoc)—OH | 3 | 15 | 2 |
| 5 | Deblock | — | 7 | 2 |
| 6 | Boc-Pra-OH | 4 | 15 | 1 |
| 7 | Boc-Pra-OH | 2 | 15 | 1 |

* Upon reaction, the mixture was gently stirred using a reciprocating shaker
* After a step was completed, solid phase was sufficiently washed with DMF and then moved to the next step
* Deblock refers to a step of deprotecting the N-terminal Fmoc group with a 20% piperidine/DMF solution
*Coupling of each amino acid was carried out at the following composition ratio (molar ratio): Protected amino acid:HATU:DIEA = 1:1:2
*The reagents were dissolved in DMF such that the amino acid solution concentration during reaction was 0.2M.

After the synthesis, D. W., TIPS, and TFA were added to 0.1 mmol of the solid phase at a ratio of D. W. (mL):TIPS (mL):TFA (mL) of 1.5:1.5:30, and the resulting mixture was stirred for 1.5 hours to perform cleavage and deprotection. After the cleavage, the solution was recovered by filtration, and then concentrated under reduced pressure, followed by adding a small amount of water and then freeze-drying. After the freeze-drying, purification was carried out by reversed-phase HPLC under the following conditions using 0.1% TFA and ACN as eluent.

Purification Conditions:

Eluent A: 0.1% TFA, Eluent B: 0.1% TFA ACN

Equilibration: Eluent A 100%, 10 mL/min, 10 min

Elution: Eluent A 100%→Eluent A 70%/Eluent B 30%, 10 mL/min, 30 min linear gradient

The purified product was subjected to mass spectrometry using MALDI-TOF MASS (under the following conditions) to confirm the product of interest.

Mass Spectrometry Conditions:

Matrix solution: 10 mg/mL α-CHCA in 0.1% TFA 50% CAN aqueous solution

Sample: HPLC eluate or 0.1% TFA 50% ACN aqueous solution (about 1 mg/mL peptide)

The matrix solution and the sample were mixed together at 1:1 to allow formation of mixed crystals on a plate.

### 3. Antigen Peptide Synthesis

Synthesis of the antigen peptide was carried out using Fmoc solid-phase synthesis similarly to the synthesis of the MAP core.

Specifically, 0.4 mmol Fmoc-Pro-TrtA-PEG-Resin was used as a solid-phase carrier. The resin was first swelled with DMF, and then the Fmoc group was deprotected with 20% piperidine. Thereafter, the synthesis was carried out by the following procedures.

The sequence of the antigen peptide was N₃-IKKADGSECLP-OH, and the peptide was extended from the C-terminus to the N-terminus.

TABLE 2

| | | Amino acid | Reaction time | |
|---|---|---|---|---|
| Step | | (mmol) | (minutes) | Times |
| 1. | Fmoc-amino acid | 1.2 | 15 | 1 |
| 2. | Step 1 and Step 2, where the amino acid(s) was/were changed in | | | |
| | accordance with the sequence, are repeated | | | |
| 3. | Deblock | — | 7 | 1 |
| 4. | N₃-PEG-COOH | 0.4 | 20 | 1 |

* After a step was completed, solid phase was sufficiently washed with DMF and then moved to the next step
* Upon reaction, the mixture was gently stirred using a reciprocating shaker
* Deblock refers to a step of deprotecting the N-terminal Fmoc group with a 20% piperidine/DMF solution
* Fmoc-amino acids used herein are as follows: Fmoc-Ala-OH; Fmoc-Cys(Trt)-OH; Fmoc-Asp(OtBu)—OH; Fmoc-Glu(OtBu)—OH; Fmoc-Gly-OH; Fmoc-Ile-OH; Fmoc-Lys(Boc)-OH; Fmoc-Ile-OH; Fmoc-Pro-OH; Fmoc-Ser(tBu)—OH.
* The amino acids were coupled with each other in the following composition ratio (molar ratio): Protected amino acid (mmol):HATU (mmol):DIEA (mmol):DMF (ml) = 1.2:1.2:2.4:8 ml

After the synthesis, thioanisole, m-cresol, TIPS, and TFA were added to 1 mmol of the solid phase at a ratio of thioanisole (mL):m-cresol (mL):TIPS (mL):TFA (mL)=3.6:1:0.6:25, and the resulting mixture was stirred for 1.5 hours to perform cleavage and deprotection. After the cleavage, the solution was recovered by filtration, and then concentrated under reduced pressure, followed by adding ether and collecting the precipitate to obtain unpurified peptide. The unpurified peptide was purified by reversed-phase HPLC under the following conditions using 0.1% TFA and ACN as eluent.

Purification Conditions:

Eluent A: 0.1% TFA, Eluent B: 0.1% TFA ACN

Equilibration: Eluent A 90%/Eluent B 10%, 10 mL/min, 10 min

Elution: Eluent A 90%/Eluent B 10%→Eluent A 50%/Eluent B 50%, 10 mL/min, 30 min linear gradient

HPLC Analysis Conditions:

Eluent A: 0.1% TFA, Eluent B: 0.1% TFA ACN

Equilibration: Eluent A 95%/Eluent B 5%, 10 mL/min, 10 min

Elution: Eluent A 90%/Eluent B 10%→Eluent A 40%/Eluent B 60%, 10 mL/min, 30 min linear gradient

The purified product was subjected to mass spectrometry using MALDI-TOF MASS (under the same conditions as described above) to confirm the product of interest.

155 mg (113 μmol) of the purified antigen peptide was dissolved in 1 mL DMSO, and 85 mg (226 μmol) BOC-Cys(Npys)-OH was added to form a disulfide, thereby to protect the SH group in the side chain containing Cys residue in the sequence. After the reaction, similarly purification was carried out by HPLC in the same manner as described above, followed by freeze-drying to obtain an antigen peptide.

### 4. Synthesis of MAP-Peptide

The MAP core was bound to the antigen peptides by utilizing the Huisgen reaction. That is, the alkynes in the MAP core were activated by Cu⁺ and allowed to react with the azide group at the N-terminus of each antigen peptide, thereby leading to binding them through triazole. The specific steps are described below.

(Step 1) The MAP core and the antigen peptide were dissolved in 0.1% aqueous TFA solution. The mixing ratio was the MAP core 15 mg (19 μmol):the antigen peptide 114 mg (71 μmol), and they were dissolved in 2 mL of an aqueous 8 M urea solution (‘peptide solution’).

(Step 2) Preparation of an aqueous copper sulfate pentahydrate solution and an aqueous ascorbic acid solution was carried out as follows. Copper sulfate pentahydrate 50 mg (200 μmol) was dissolved in 1 mL D. W. (‘aqueous copper sulfate solution’). Ascorbic acid was dissolved in 1 mL D. W., 176 mg (1 mmol) (‘aqueous ascorbic acid solution’). Subsequently, the total amount of the aqueous copper sulfate solution and the total amount of the aqueous ascorbic acid solution were admixed (‘Cu⁺ solution’).

(Step 3) Subsequently, Huisgen reaction was carried out. Specifically, the peptide solution 2 mL and the Cu⁺ solution 0.35 mL were admixed to allow to react them at room temperature for several hours.

(Step 4) The reaction product was subjected to reversed-phase HPLC using 0.1% TFA and ACN as eluent, and all peaks that appeared near the antigen peptide were collected, followed by freeze-drying (recovery, 87 mg).

(Step 5) In 1 mL of 1/15 M phosphate buffer (K/Na₂, pH 7.2), 87 mg of the freeze-dried sample obtained as described above was dissolved, and the pH was adjusted to a neutral pH with 4% sodium hydrogen carbonate solution. To this solution, 34 mg (220 μmol) dithiothreitol was added to perform reduction at room temperature, and then the product of interest was purified by reversed-phase HPLC. The purification conditions were the same as those for the purification of the antigen peptide.

(Step 6) Mass spectrometry using MALDI-TOF MASS was carried out (under the same conditions as described above) to confirm the product of interest. HPLC analysis was carried out to determine the purity. The HPLC purity-determining conditions were the same as those for the antigen peptide.

### 5. Synthesis Results

The synthesis results were as follows.

TABLE 3
MAP core

| | | Theoretical | | Yield | | |
|---|---|---|---|---|---|---|
| Name | Sequence | mg | μmol | mg | μmol | Yield (%) |
| MAP4 | Pra₄—K₂—K—NH₂ | 782 | 1000 | 501 | 641 | 64% |
| core | | | | | | |

TABLE 4
Ebola 1 MAP4

| | | Theoretical | | Yield | | Yield (%) |
|---|---|---|---|---|---|---|
| Name | Sequence | mg | μmol | mg | μmol | (Step) |
| Antigen | N₃-PEG- | 550 | 400 | 155 | 113 | 28% |
| peptide | IKKADGSECLP | | | | | |
| | N₃-PEG- | 180 | 113 | 114 | 71 | 63% |
| | IKKADGSEC | | | | | |
| | (Boc-Cys-OH)LP | | | | | |
| Ebola 1 | Core-[PEG- | 128 | 18 | 87 | 12 | 68% |
| SS-MAP4 | IKKADGSEC | | | | | |
| | (Boc-Cys-OH)LP]₄ | | | | | |
| Ebola 1 | Core-[PEG- | 76 | 12 | 37 | 6 | 48% |
| MAP4 | IKKADGSECLP]₄ | | | | | |

### <Synthesis of Ebola 2 MAP4>

### 1. Abbreviations

NH2-SAL-Trt(2-Cl)-Resin: Rink-Bernatowitz-amide Barlos Resin (Watanabe Chemical Industries, Ltd.)

Fmoc-Lys(Fmoc)-OH: N-α,N-ε-Bis(9-fluorenylmethoxycarbonyl)-L-lysine (Watanabe Chemical Industries, Ltd.)

Boc-Pra-OH: N-Boc-L-propargylglycine (Tokyo Chemical Industry Co., Ltd.)

N₃-PEG-COOH: 11-Azido-3,6,9-trioxaundecanoic Acid (Tokyo Chemical Industry Co., Ltd.)

Fmoc-Lys(Boc)-Alko-PEG resin: N-α-(9-Fluorenylmethoxycarbonyl)-N-ε-(t-butoxycarbonyl)-L-lysine p-methoxybenzyl alcohol polyethyleneglycol resin (Watanabe Chemical Industries, Ltd.)

Fmoc-Cys(Trt)-OH: N-α-(9-Fluorenylmethoxycarbonyl)-S-trityl-L-cysteine (Watanabe Chemical Industries, Ltd.)

Fmoc-Phe-OH: N-α-(9-Fluorenylmethoxycarbonyl)-L-phenylalanine (Watanabe Chemical Industries, Ltd.)

Fmoc-His(Trt)-OH: N-α-(9-Fluorenylmethoxycarbonyl)-N-τ-trityl-L-histidine (Watanabe Chemical Industries, Ltd.)

Fmoc-Pro-OH: N-(9-Fluorenylmethoxycarbonyl)-L-proline (Watanabe Chemical Industries, Ltd.)

Fmoc-Arg(Pbf)-OH: N-α-(9-Fluorenylmethoxycarbonyl)-N-ω-(2,2,4,6,7-pentamethyldihydrobenzofuran-5-sulfonyl)-L-arginine (Watanabe Chemical Industries, Ltd.)

Fmoc-Val-OH: N-α-(9-Fluorenylmethoxycarbonyl)-L-valine (Watanabe Chemical Industries, Ltd.)

Fmoc-Tyr(tBu)-OH: N-α-(9-Fluorenylmethoxycarbonyl)-O-(t-butyl)-L-tyrosine (Watanabe Chemical Industries, Ltd.)

Boc-Cys(Npys)-OH: N-α-(t-Butoxycarbonyl)-S-(3-nitro-2-pyridinesulfenyl)-L-cysteine (Kokusan Chemical Co., Ltd.)

HATU: O-(7-aza-1H-benzotriazol-1-yl)-N,N,N′,N′-tetramethyluronium hexafluorophosphate (Genscript)

DIEA: N,N-Diisopropylethylamine (Wako Pure Chemical Industries, Ltd.; for peptide synthesis)

DMF: N,N-Dimethylformamide (Kanto Chemical Co., Inc.; for peptide synthesis)

TFA: 2,2,2-Trifluoroacetic acid (Wako Pure Chemical Industries, Ltd.)

TIPS: Triisopropylsilane (Watanabe Chemical Industries, Ltd.)

Thioanisole (Watanabe Chemical Industries, Ltd.)

m-Cresol (Tokyo Chemical Industry Co., Ltd.)

DCM: Dichloromethane (Kanto Chemical Co., Inc.)

ACN: Acetonitrile (Kanto Chemical Co., Inc.; for HPLC)

α-CHCA: α-Cyano-4-hydroxycinnamic acid

Preparative column: YMC-Pack Pro C18; 20 mm (I. D.)×250 mm (length); particle size, 5 μm; pore size, 12 μm (YMC)

Analytical column: YMC-Pack Pro C18; 4.6 mm (I. D.)×250 mm (length); particle size, 5 μm; pore size, 12 μm (YMC)

MALDI-TOF MASS: Matrix Assisted Laser Desorption Ionization Time Of Flight Mass Spectrometry

D. W.: Distilled water

Copper sulfate pentahydrate (Kanto Chemical Co., Inc.)

Ascorbic acid (Kanto Chemical Co., Inc.)

### 2. Synthesis of MAP Core

Using MAP4 as an example, a synthesis method for the MAP core is described below.

For the synthesis of the MAP core, conventional Fmoc solid-phase synthesis was employed, and all steps were manually carried out. Specifically, 1 mmol NH2-SAL-Trt(2-Cl)-Resin was used as a solid-phase carrier to perform the synthesis by the following procedures.

TABLE 5

| | | Amino acid | Reaction Time | |
|---|---|---|---|---|
| | Step | (mmol) | (minutes) | Times |
| 1 | Deblock | — | 7 | 1 |
| 2 | Fmoc-Lys(Fmoc)—OH | 3 | 15 | 1 |
| 3 | Deblock | — | 7 | 1 |
| 4 | Fmoc-Lys(Fmoc)—OH | 3 | 15 | 2 |
| 5 | Deblock | — | 7 | 2 |
| 6 | Boc-Pra-OH | 4 | 15 | 1 |
| 7 | Boc-Pra-OH | 2 | 15 | 1 |

* Upon reaction, the mixture was gently stirred using a reciprocating shaker
* After a step was completed, solid phase was sufficiently washed with DMF and then moved to the next step
* Deblock refers to a step of deprotecting the N-terminal Fmoc group with a 20% piperidine/DMF solution
*Coupling of each amino acid was carried out at the following composition ratio (molar ratio): Protected amino acid:HATU:DIEA = 1:1:2
*The reagents were dissolved in DMF such that the amino acid solution concentration during reaction was 0.2M.

After the synthesis, D. W., TIPS, and TFA were added to 0.1 mmol of the solid phase at a ratio of D. W. (mL):TIPS (mL):TFA (mL) of 1.5:1.5:30, and the resulting mixture was stirred for 1.5 hours to perform cleavage and deprotection. After the cleavage, the solution was recovered by filtration, and then concentrated under reduced pressure, followed by adding a small amount of water and then freeze-drying. After the freeze-drying, purification was carried out by reversed-phase HPLC under the following conditions using 0.1% TFA and ACN as eluent.

Purification Conditions:

Eluent A: 0.1% TFA, Eluent B: 0.1% TFA ACN

Equilibration: Eluent A 100%, 10 mL/min, 10 min

Elution: Eluent A 100%→Eluent A 70%/eluent B 30%, 10 mL/min, 30 min linear gradient The purified product was subjected to mass spectrometry using MALDI-TOF MASS (under the following conditions) to confirm the product of interest.

Mass Spectrometry Conditions:

Matrix solution: 10 mg/mL α-CHCA in 0.1% TFA 50% CAN aqueous solution

Sample: HPLC eluate or 0.1% TFA 50% ACN aqueous solution (about 1 mg/mL peptide)

The matrix solution and the sample were admixed at 1:1 to form mixed crystals on a plate.

### 3. Antigen Peptide Synthesis

Synthesis of the antigen peptide was carried out using Fmoc solid-phase synthesis similarly to the synthesis of the MAP core.

Specifically, 0.4 mmol Fmoc-Lys(Boc)-Alko-PEG-Resin was used as a solid-phase carrier. The resin was first swelled with DMF, and then the Fmoc group was deprotected with 20% piperidine. Thereafter, the synthesis was carried out by the following procedures. The sequence of the antigen peptide was N₃-PEG-FPRCRYVHK-OH, and the peptide was extended from the C-terminus to the N-terminus.

TABLE 6

| | | Amino acid | Reaction time | |
|---|---|---|---|---|
| Step | | (mmol) | (minutes) | Times |
| 1. | Fmoc-amino acid | 1.2 | 15 | 1 |
| 2. | Step 1 and Step 2, where the amino acid(s) was/were changed in | | | |
| | accordance with the sequence, are repeated | | | |
| 3. | Deblock | — | 7 | 1 |
| 4. | N₃-PEG-COOH | 0.4 | 20 | 1 |

* After a step was completed, solid phase was sufficiently washed with DMF and then moved to the next step
* Upon reaction, the mixture was gently stirred using a reciprocating shaker
* Deblock refers to a step of deprotecting the N-terminal Fmoc group with a 20% piperidine/DMF solution
* Fmoc-amino acids used herein are as follows: Fmoc-Cys(Trt)-OH; Fmoc-Phe-OH; Fmoc-His(Trt)-OH; Fmoc-Pro-OH; Fmoc-Arg(Pbf)-OH; Fmoc-Val-OH; Fmoc-Tyr(tBu)—OH.
* The amino acids were coupled with each other in the following composition ratio (molar ratio): Protected amino acid (mmol):HATU (mmol):DIEA (mmol):DMF (ml) = 1.2:1.2:2.4:8 ml

After the synthesis, thioanisole, m-cresol, TIPS, and TFA were added to 1 mmol of the solid phase at a ratio of thioanisole (mL):m-cresol (mL):TIPS (mL):TFA (mL)=3.6:1:0.6:25, and the resulting mixture was stirred for 1.5 hours to perform cleavage and deprotection. After the cleavage, the solution was recovered by filtration, and then concentrated under reduced pressure. Ether was further added, and the precipitate was collected to obtain unpurified peptide. The unpurified peptide was purified by reversed-phase HPLC under the following conditions using 0.1% TFA and ACN as eluent.

Purification Conditions:

Eluent A: 0.1% TFA, Eluent B: 0.1% TFA ACN

Equilibration: Eluent A 90%/Eluent B 10%, 10 mL/min, 10 min

Elution: Eluent A 90%/Eluent B 10%→Eluent A 50%/Eluent B 50%, 10 mL/min, 30 min linear gradient

### HPLC Analysis Conditions:

Eluent A: 0.1% TFA, Eluent B: 0.1% TFA ACN

Equilibration: Eluent A 90%/Eluent B 90%, 10 mL/min, 10 min

Elution: Eluent A 90%/Eluent B 10%→Eluent A 40%/Eluent B 60%, 10 mL/min, 30 min linear gradient

The purified product was subjected to mass spectrometry using MALDI-TOF MASS (under the same conditions as described above) to confirm the product of interest.

The purified antigen peptide 370 mg (260 μmol) was dissolved in 2 mL DMSO, and 220 mg (586 μmol) BOC-Cys(Npys)-OH was added to form a disulfide, thereby to protect the SH group in the side chain containing Cys residue in the sequence. After the reaction, similarly purification was carried out by HPLC in the same manner as described above, followed by freeze-drying to obtain an antigen peptide.

### 4. Synthesis of MAP-Peptide

The MAP core was bound to the antigen peptides by utilizing the Huisgen reaction. That is, the alkyne in the MAP core was activated by Cu⁺ to allow it to react with the azide group at the N-terminus of each antigen peptide, thereby binding them through triazole. Specific steps are described below.

(Step 1) The MAP core and the antigen peptide were dissolved in 0.1% aqueous TFA solution. The mixing ratio was 40 mg (51 μmol) of the MAP core:320 mg (195 μmol) of the antigen peptide, and they were dissolved in 2 mL aqueous 8 M urea solution (‘peptide solution’).

(Step 2) Preparation of an aqueous copper sulfate pentahydrate solution and an aqueous ascorbic acid solution was carried out as follows. 250 mg (500 μmol) copper sulfate pentahydrate was dissolved in 1 mL D. W. (‘aqueous copper sulfate solution’). 440 mg (2.5 mmol) ascorbic acid was dissolved in 1 mL D. W. (‘aqueous ascorbic acid solution’). Subsequently, the total amount of aqueous copper sulfate solution and the total amount of aqueous ascorbic acid solution were admixed (‘Cu⁺ solution’).

(Step 3) Subsequently, Huisgen reaction was performed. Specifically, 2 mL of the peptide solution and 1 ml of the Cu⁺ solution were admixed to allow them to react at room temperature for several hours.

(Step 4) The reaction product was subjected to reversed-phase HPLC using 0.1% TFA and ACN as eluent, and all peaks that appeared near the antigen peptide were collected, followed by freeze-drying (recovery, 273 mg).

(Step 5) In 1 mL of 1/15 M phosphate buffer (K/Na₂) pH 7.2, 273 mg of the freeze-dried sample obtained as described above was dissolved, and the pH was adjusted to a neutral pH with 4% sodium hydrogen carbonate solution. To this solution, 68 mg (440 μmol) dithiothreitol was added to perform reduction at room temperature, and then the product of interest was purified by reversed-phase HPLC. The purification conditions were the same as those for the purification of the antigen peptide.

(Step 6) Mass spectrometry using MALDI-TOF MASS was carried out (under the same conditions as described above) to confirm the product of interest. HPLC analysis was carried out to determine the purity. The HPLC purity-determining conditions were the same as those for the antigen peptide.

### 5. Synthesis Results

The synthesis results were as follows.

TABLE 7
MAP core

| | | Theoretical | | Yield | | |
|---|---|---|---|---|---|---|
| Name | Sequence | mg | μmol | mg | μmol | Yield (%) |
| MAP4 | Core | 782 | 1000 | 501 | 641 | 64% |
| | Pra₄—K₂—K—NH₂ | | | | | |

TABLE 8
Ebola 2 MAP4

| | | Theoretical | | Yield | | Yield (%) |
|---|---|---|---|---|---|---|
| Name | Sequence | mg | μmol | mg | μmol | (Step) |
| Antigen | N₃-PEG- | 568 | 400 | 370 | 260 | 65% |
| peptide | FPRCRYVHK | | | | | |
| | N₃-PEG-FPRC | 426 | 260 | 320 | 195 | 75% |
| | (Boc-Cys-OH) | | | | | |
| | RYVHK | | | | | |
| Ebola 2_ | Core-[PEG-FPRC | 358 | 49 | 273 | 37 | 76% |
| SS-MAP4 | (Boc-Cys-OH) | | | | | |
| | RYVHK]₄ | | | | | |
| Ebola 2_ | Core-[PEG- | 239 | 37 | 210 | 32 | 88% |
| MAP4 | FPRCRYVHK]₄ | | | | | |

### Example 2

### <Administration Procedure for Ebola 1 MAP4 or Ebola 2 MAP4>

### (1) Test A

Mouse Group 1, Group 2, and Group 3 were provided for different doses (100 μg, 10 μg, or 1 μg in 10% mouse serum) of Ebola 1 MAP4 or Ebola 2 MAP4 (referred to as “Ebola-MAP4”). As a control for the serum addition groups, Mouse Group 4 to which 100 μg Ebola-MAP4 in physiological saline was administered, was provided. The mice were BALB/cAJc (CLEA Japan, Inc.) mice (8-weeks old, female), and each group consisted of five animals.

To each of the mice in Group 1 to Group 4, α-galactosylceramide (2 μg) and Ebola-MAP4 were intravenously administered only for the initial administration (Day 0), and then Ebola-MAP4 alone was administered on Day 1, Day 3, Day 7, and Day 14. Three days before the initial administration, and on Day 1, Day 7, Day 14, and Day 21 after the initial administration, orbital blood was drawn under anesthesia with isoflurane, and the concentration of anti-MAP antibody in sera was measured.

### (2) Test B

To Balb/c mice, Ebola 1 MAP4 dissolved in physiological saline containing 2% DMSO and 1% mouse serum was intraperitoneally injected. The dose was 100 μg/100 μL/mouse/administration per Balb/c mouse. Regarding the procedure of administration of the MAP, the administration was carried out once daily for five continuous days (five times of administration in total), and then administration was carried out on Day 7 and Day 14 after the day of initial administration. Administration of α-galactosylceramide was carried out by intraperitoneally administering together with MAP4 only for the initial administration, and the dose was 2 gig/mouse. Before and after the administration, blood was drawn from the orbital venous plexus, and the concentration of anti-MAP antibody in sera was measured.

### (3) Test C

A mixture of Ebola 1 MAP4 and Ebola 2 MAP4 dissolved in 2% DMSO-PBS was intraperitoneally injected into BDF1 mice. The dose was 200 μg/100 μL/mouse/administration in terms of the total amount of MAP4 per mouse. Regarding the procedure of administration of the MAP, the administration was carried out once daily for five continuous days (five times of administration in total), and then administration was carried out on Day 7 and Day 14 after the day of initial administration. Administration of α-galactosylceramide was carried out by intraperitoneally administering together with MAP4 only for the initial administration, and the dose was 2 μg/mouse. Before and after the administration, blood was drawn from the orbital venous plexus, and the concentration of anti-MAP antibody in sera was measured.

### <Method for Measuring Antibody Titer for Ebola-MAP4 in Mouse Serum>

For measurement of anti-MAP antibodies, Ebola 1 or Ebola 2 peptides to which bovine serum albumin (BSA) and FLAG were bound were immobilized on an ELISA plate(s), and then 100-fold diluted serum was added thereto, followed by incubation at 37° C. for 1 hour. Thereafter, to each sample, peroxidase-labeled anti-IgM antibody (SouthernBiotech), anti-IgG antibody (SouthernBiotech), anti-IgG1 antibody (SouthernBiotech), anti-IgG2a antibody (SouthernBiotech), and anti-IgG3 antibody (SouthernBiotech) were added at the concentrations recommended by the manufacturer, and color development after degradation of the substrate by peroxidase was measured using a plate reader to determine antibody titers in the sera.

In addition, the Ebola 1 or Ebola 2 peptide to which bovine serum albumin (BSA) and FLAG were bound was immobilized on an ELISA plate, and, instead of the serum, an anti-FLAG monoclonal antibody (Clone: M2 mouse IgG1, Sigma-Aldrich) diluted to various concentrations was added thereto, followed by measurement in the same manner as the measurement of the anti-MAP antibody titer, to provide a standard curve for quantification of the anti-MAP antibody titer. From this standard curve, approximate antibody concentrations of anti-MAP antibodies in sera were calculated.

### <Results of Test A>

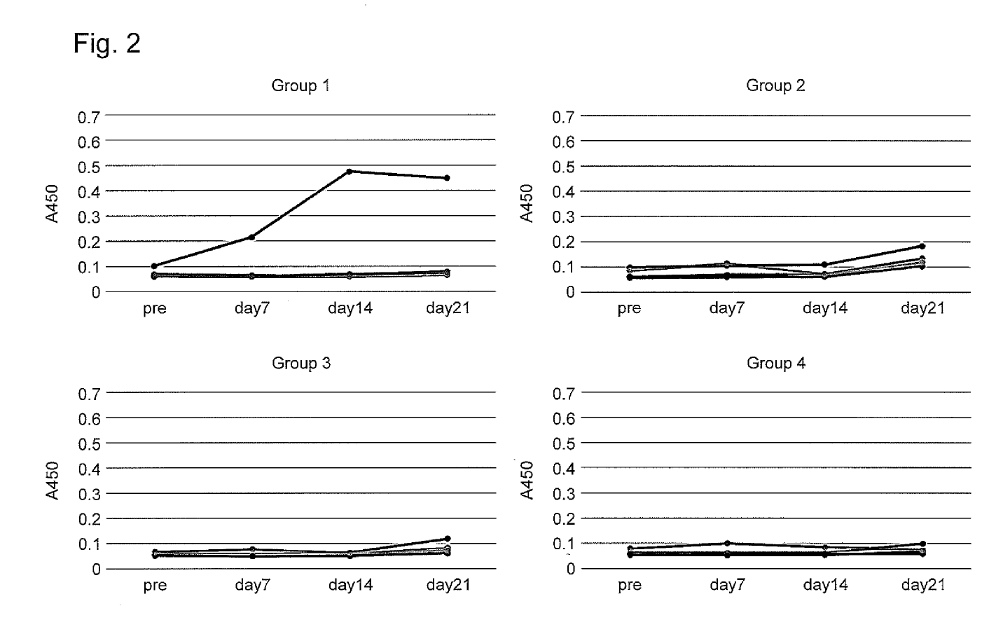

The results of measurement of the concentrations of anti-MAP antibodies in the sera are shown in FIG. 2.

From FIG. 2, in the intravenous administration of Ebola 1 MAP4, increase in IgG was seen only in the 100-μg administration group.

However, no increase in IgG or IgM was seen in the intravenous administration of Ebola 2 MAP4.

### <Results of Test B>

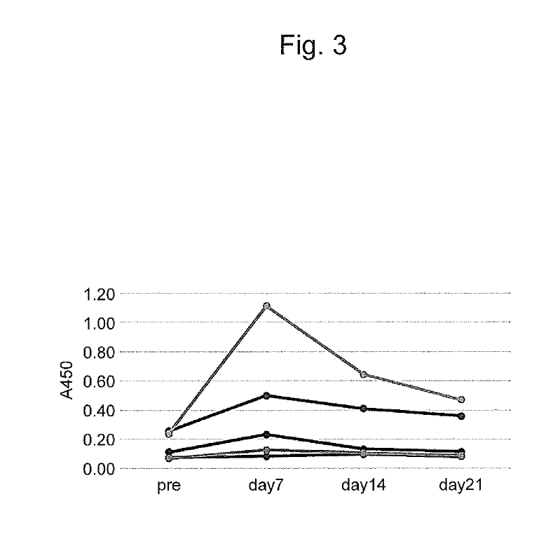

The results of measurement of the concentrations of anti-MAP antibodies in the sera are shown in FIG. 3.

From FIG. 3, among the mice tested, one mouse showed an evident increase, and two mice showed mild increases in IgM in the intraperitoneal administration of Ebola 1 MAP4. However, no increase in IgG was seen.

### <Results of Test C>

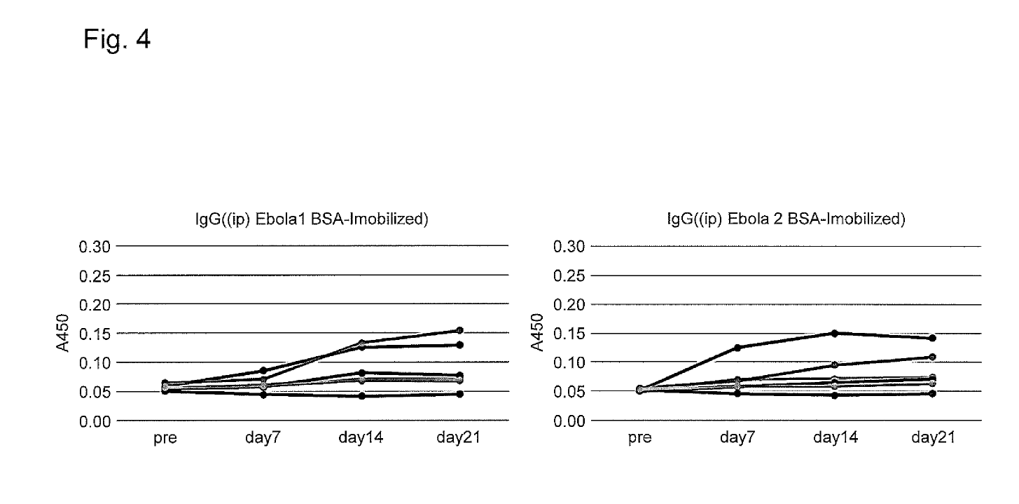

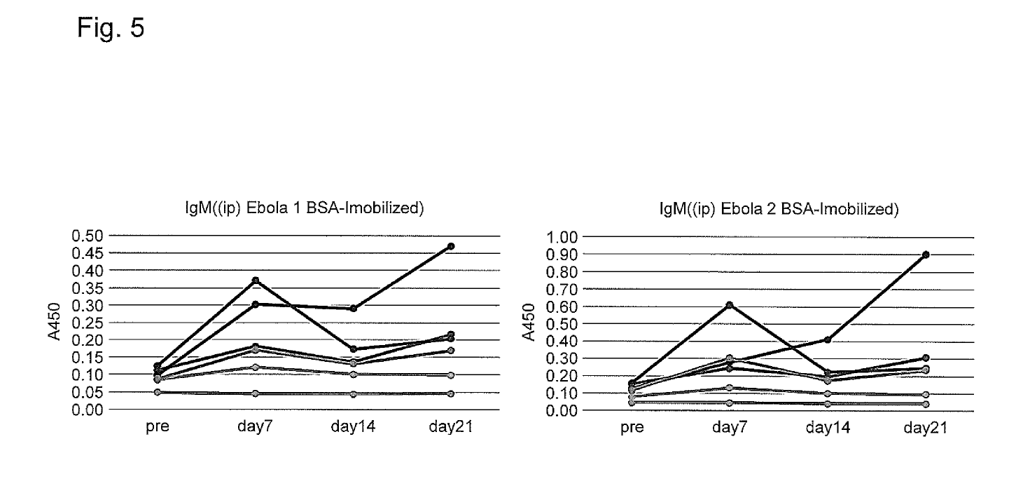

The results of measurement of the concentrations of anti-MAP antibodies in the sera are shown in FIG. 4 (IgG titer) and FIG. 5 (IgM titer).

From FIG. 4, among the mice tested, as a result of measurement of the IgG titers against Ebola 1 (left panel) and Ebola 2 (right panel) in the mixed administration of Ebola 1 MAP4 and Ebola 2 MAP4, two mice showed increases in IgG against Ebola 1, and, similarly two mice showed increases in IgG against Ebola 2. The Ebola 1-IgG concentrations measured were 10 to 20 ng/mL.

From FIG. 5, among the mice tested, two mice showed evident increases, and two mice showed mild increases in the IgM titers against Ebola 1 (left panel) and Ebola 2 (right panel).

### INDUSTRIAL APPLICABILITY

The present invention provides an immune inducer against Ebola virus infection, for which no practical therapeutic methods or vaccines have been available. As shown in Examples, induction of IgG antibodies was enabled by said multiple antigen peptides, indicating a possibility of vaccines against Ebola virus prepared by the immune induction. This is industrially useful for prevention and treatment of Ebola virus infection, which is highly lethal.

All publications, patents, and patent applications cited herein are incorporated herein by reference in their entirety.

1. A multiple antigen peptide comprising a dendritic core and 4-8 antigen peptides, wherein each of the antigen peptides is bound to a terminus of the dendritic core directly or through a spacer and is a peptide consisting of 7-15 consecutive amino acids in the amino acid sequence of SEQ ID NO: 1 or a peptide which is the same as the peptide except that 1-3 amino acids are substituted.

2. The multiple antigen peptide according to claim 1, wherein the peptide is a peptide consisting of 7-15 consecutive amino acids in the amino acid sequences of SEQ ID NOs: 8 to 12, or a peptide which is the same as the peptide except that 1-3 amino acids are substituted.

3. The multiple antigen peptide according to claim 1, wherein the peptide is a peptide consisting of 7-15 consecutive amino acids in the amino acid sequence of SEQ ID NO: 2, SEQ ID NO: 3, or SEQ ID NO: 4, or a peptide which is the same as the peptide except that 1-3 amino acids are substituted.

4. The multiple antigen peptide according to claim 1, wherein the peptide is a peptide consisting of 7-11 consecutive amino acids in the amino acid sequence of SEQ ID NO: 5, a peptide consisting of 7 or 8 consecutive amino acids in the amino acid sequence of SEQ ID NO: 32, or a peptide consisting of 7-9 consecutive amino acids in the amino acid sequence of SEQ ID NO: 6, or a peptide which is the same as one of the peptides except that 1-3 amino acids are substituted.

5. The multiple antigen peptide according to claim 1, wherein all of the antigen peptides are peptides having an identical amino acid sequence.

6. The multiple antigen peptide according to claim 1, wherein the dendritic core comprises a plurality of lysine residues.

7. The multiple antigen peptide according to claim 6, wherein the dendritic core further comprises a cysteine residue.

8. The multiple antigen peptide according to claim 1, wherein the spacer comprises a polyoxyalkylene chain.

9. The multiple antigen peptide according to claim 1, wherein the multiple antigen peptide is characterized by being represented by the following Formula (I): 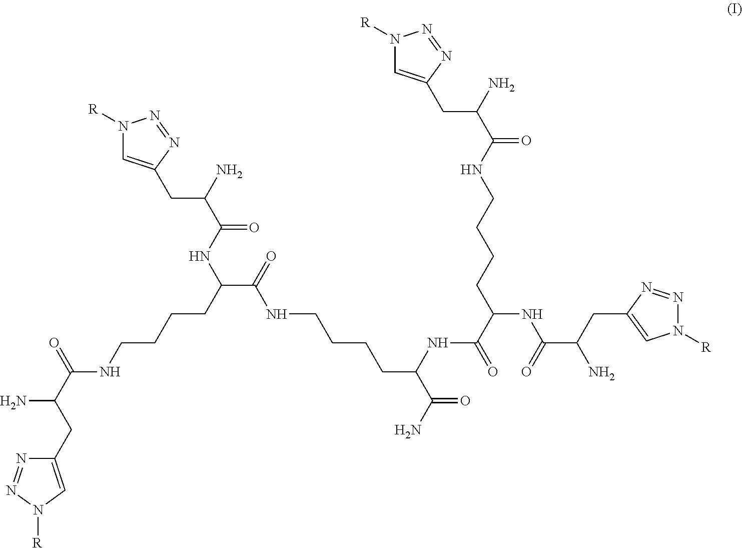
where R is: 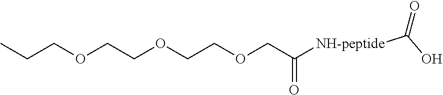
where the peptide represents an antigen peptide.

10. An immune inducer comprising one or at least two multiple antigen peptides according to claim 1 as the active ingredient.

11. The immune inducer according to claim 10, further comprising an adjuvant having an ability to produce interferon γ.

12. The immune inducer according to claim 11, wherein the adjuvant is α-galactosylceramide or an analog thereof.

13. The immune inducer according to claim 10, which is for use in treatment or prevention of Ebola virus infection in a mammal.

14. The immune inducer according to claim 10, comprising a pharmaceutically acceptable carrier.

15. A method for treatment or prevention of Ebola virus infection in a mammal, comprising administering the immune inducer according to claim 10 to the mammal.


SEQUENCE LISTING (38 sequences)

SEQ ID NO: 1 - protein, 38 aa, Ebola virus
  misc_feature (3)..(3): Xaa = Glu, Ala, or Asp
  misc_feature (7)..(7): Xaa = Pro, Ser, Val, or Ala
  misc_feature (15)..(15): Xaa = Ala, Pro, Leu, or Glu
  misc_feature (16)..(16): Xaa = Ala or Pro
  misc_feature (18)..(18): Xaa = Asp or Glu
  misc_feature (20)..(20): Xaa = Ile or Val
  misc_feature (22)..(22): Xaa = Gly or Asp
  misc_feature (32)..(32): Xaa = Val or Ala
  misc_feature (33)..(33): Xaa = Ser or Gln
NLXIKKXDGSECLPXXPXGXRXFPRCRYVHKXXGTGPC

SEQ ID NO: 2 - protein, 17 aa, Ebola virus
  misc_feature (3)..(3): Xaa = Glu, Ala or Asp
  misc_feature (7)..(7): Xaa = Pro, Ser, Val or Ala
  misc_feature (15)..(15): Xaa = Ala, Pro, Leu or Glu
  misc_feature (16)..(16): Xaa = Ala or Pro
NLXIKKXDGSECLPXXP

SEQ ID NO: 3 - protein, 18 aa, Ebola virus
  misc_feature (2)..(2): Xaa = Asp or Glu
  misc_feature (4)..(4): Xaa = Ile or Val
  misc_feature (6)..(6): Xaa = Gly or Asp
  misc_feature (16)..(16): Xaa = Val or Ala
  misc_feature (17)..(17): Xaa = Ser or Gln
PXGXRXFPRCRYVHKXXG

SEQ ID NO: 4 - protein, 18 aa, Ebola virus
  misc_feature (2)..(2): Xaa = Gly or Asp
  misc_feature (12)..(12): Xaa = Val or Ala
  misc_feature (13)..(13): Xaa = Ser or Gln
RXFPRCRYVHKXXGTGPC

SEQ ID NO: 5 - protein, 11 aa, Ebola virus
  misc_feature (4)..(4): Xaa = Pro, Ser, Val or Ala
IKKXDGSECLP

SEQ ID NO: 6 - protein, 9 aa, Ebola virus
FPRCRYVHK

SEQ ID NO: 7 - protein, 676 aa, Ebola virus
MGVTGILQLPRDRFKRTSFFLWVIILFQRTFSIPLGVIHNSTLQVSDVDKLVCRDKLSSTNQLRSVGLNLEGNGVATDVPSVTKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNEAEYLFEVDNLTYVQLESRFTPQFLLQLNETIYASGKRSNTTGKLIWKVNPEIDTTIGEWAFWETKKNLTRKIRSEELSFTAVSNGPKNISGQSPARTSSDPETNTTNEDHKIMASENSSAMVQVHSQGRKAAVSHLTTLATISTSPQPPTTKTGPDNSTHNTPVYKLDISEATQVGQHHRRADNDSTASDTPPATTAAGPLKAENTNTSKSADSLDLATTTSPQNYSETAGNNNTHHQDTGEESASSGKLGLITNTIAGVAGLITGGRRTRREVIVNAQPKCNPNLHYWTTQDEGAAIGLAWIPYFGPAAEGIYTEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 8 - protein, 38 aa, Ebola virus
NLEIKKPDGSECLPAAPDGIRGFPRCRYVHKVSGTGPC

SEQ ID NO: 9 - protein, 38 aa, Ebola virus
NLEIKKPDGSECLPPPPDGVRGFPRCRYVHKAQGTGPC

SEQ ID NO: 10 - protein, 38 aa, Ebola virus
NLEIKKSDGSECLPLPPDGVRGFPRCRYVHKVQGTGPC

SEQ ID NO: 11 - protein, 38 aa, Ebola virus
NLAIKKVDGSECLPEAPEGVRDFPRCRYVHKVSGTGPC

SEQ ID NO: 12 - protein, 38 aa, Ebola virus
NLDIKKADGSECLPEAPEGVRGFPRCRYVHKVSGTGPC

SEQ ID NO: 13 - protein, 17 aa, Ebola virus
NLEIKKPDGSECLPAAP

SEQ ID NO: 14 - protein, 17 aa, Ebola virus
NLEIKKPDGSECLPPPP

SEQ ID NO: 15 - protein, 17 aa, Ebola virus
NLEIKKSDGSECLPLPP

SEQ ID NO: 16 - protein, 17 aa, Ebola virus
NLAIKKVDGSECLPEAP

SEQ ID NO: 17 - protein, 17 aa, Ebola virus
NLDIKKADGSECLPEAP

SEQ ID NO: 18 - protein, 18 aa, Ebola virus
PDGIRGFPRCRYVHKVSG

SEQ ID NO: 19 - protein, 18 aa, Ebola virus
PDGVRGFPRCRYVHKAQG

SEQ ID NO: 20 - protein, 18 aa, Ebola virus
PDGVRGFPRCRYVHKVQG

SEQ ID NO: 21 - protein, 18 aa, Ebola virus
PEGVRDFPRCRYVHKVSG

SEQ ID NO: 22 - protein, 18 aa, Ebola virus
PEGVRGFPRCRYVHKVSG

SEQ ID NO: 23 - protein, 18 aa, Ebola virus
RGFPRCRYVHKVSGTGPC

SEQ ID NO: 24 - protein, 18 aa, Ebola virus
RGFPRCRYVHKAQGTGPC

SEQ ID NO: 25 - protein, 18 aa, Ebola virus
RGFPRCRYVHKVQGTGPC

SEQ ID NO: 26 - protein, 18 aa, Ebola virus
RDFPRCRYVHKVSGTGPC

SEQ ID NO: 27 - protein, 18 aa, Ebola virus
RGFPRCRYVHKVSGTGPC

SEQ ID NO: 28 - protein, 11 aa, Ebola virus
IKKPDGSECLP

SEQ ID NO: 29 - protein, 11 aa, Ebola virus
IKKSDGSECLP

SEQ ID NO: 30 - protein, 11 aa, Ebola virus
IKKVDGSECLP

SEQ ID NO: 31 - protein, 11 aa, Ebola virus
IKKADGSECLP

SEQ ID NO: 32 - protein, 8 aa, Ebola virus
  misc_feature (2)..(2): Xaa = Asp or Glu
  misc_feature (4)..(4): Xaa = Ile or Val
  misc_feature (6)..(6): Xaa = Gly or Asp
PXGXRXFP

SEQ ID NO: 33 - protein, 8 aa, Ebola virus
PDGIRGFP

SEQ ID NO: 34 - protein, 8 aa, Ebola virus
PDGVRGFP

SEQ ID NO: 35 - protein, 8 aa, Ebola virus
PEGVRDFP

SEQ ID NO: 36 - protein, 8 aa, Ebola virus
PEGVRGFP

SEQ ID NO: 37 - protein, 11 aa, Ebola virus
  misc_feature (9)..(9): Xaa is Cys modified by Boc-Cys via disulfide linkage
IKKADGSEXLP

SEQ ID NO: 38 - protein, 9 aa, Ebola virus
  misc_feature (4)..(4): Xaa is Cys modified by Boc-Cys via disulfide linkage
FPRXRYVHK